(19) United States
(12) Patent Application Publication
(10) Pub. No.: US 2019/0274617 A1
(43) Pub. Date: Sep. 12, 2019

(54) BREAST CANCER DETECTION USING NEAR-FIELD PROBES WITH MACHINE LEARNING TECHNIQUES

(71) Applicant: Wave Intelligence Inc., Waterloo (CA)

(72) Inventors: Omar M. RAMAHI, Waterloo (CA); Maged A. Aldhaeebi, Waterloo (CA); Thamer S. Almoneef, Waterloo (CA)

(73) Assignee: Wave Intelligence Inc., Waterloo (CA)

(21) Appl. No.: 16/283,204

(22) Filed: Feb. 22, 2019

Related U.S. Application Data
(60) Provisional application No. 62/634,887, filed on Feb. 25, 2018.

(51) Int. Cl. A61B 5/00; A61B 5/05; A61B 6/00; G06N 7/02

(52) U.S. Cl. CPC A61B 5/4312; G06N 7/02; A61B 6/502; A61B 5/0507

(57) ABSTRACT

The present invention is a near-field microwave sensing modality that uses a single probe combined with a classification algorithm to help in detecting the presence of tumors in the human female breast. The concept employs a near-field resonant probe with an ultra-narrow frequency response. The resonant probe is highly sensitive to the changes in the electromagnetic properties of the breast tissues such that the presence of the tumor is gauged by determining the changes in the magnitude and phase response of the sensor's reflection coefficient. A key feature of our proposed detection concept is the simultaneous sensing of tissue property changes to the two female breasts since the right and left healthy breasts are morphologically and materially identical. Once the probe response is recorded for both breasts, the Principle Component Analysis (PCA) method is employed to emphasize the difference in the probe responses.

### RELATED APPLICATION

The present invention requests a priority date of the provisional patent application No. 62/634,887 filed on Feb. 25, 2018.

### FIELD OF THE INVENTION

The present invention relates in general to breast cancer detection and in specific to breast cancer detection using near-field probes with machine learning techniques.

### BACKGROUND OF THE INVENTION

Breast cancer is one of the most common types of cancer among women and it is the second leading cause of death from cancer in women worldwide. In 2017, the American Cancer Society reported that more than 40,000 women will die from breast cancer in the US. In addition, it is expected that more than 250,000 new cases of invasive female breast cancer patients will be diagnosed in the US.¹ Detection of breast cancer tumors in their early stage (when they are small and have not spread), is critical for possible successful treatment.¹ X-ray mammography, magnetic resonance imaging (MRI), and ultrasound scanning are the most common clinical imaging modalities currently used for diagnosis and detection of breast cancer.² Screening x-ray mammogram for an abnormality detection requires further investigation to confirm if cancer is present because of a high false negative rate ranging between 4 and 34%, or a high false positive rate reaching 70%.^{3,4} Moreover, x-ray has other disadvantages such as ionization which poses a health risk, and compression of the breast tissues which causes significant discomfort to women.⁵ Ultrasound uses high-frequency waves transmitted from a transducer through the breast tissues to form an image of the breast. Ultrasound provides a high contrast resolution; however, it lacks the spatial resolution of conventional mammography.⁶ MRI is highly effective for imaging small abnormalities compared with mammography and ultrasound, and can be used effectively for women with dense breasts. However, MRI testing is very expensive and the technology is not widely available in most parts of the world.⁷

Microwaves imaging (MI) and detection modalities present an attractive alternative to the available clinical detection techniques for the primary reason that it requires inexpensive technology and is non-ionizing. The core principle behind all MI modalities is the contrast in the dielectric properties between normal and malignant breast tissues. Therefore, the significant differentiation in the dielectric properties of normal and malignant breast tissues can be used as the underlying principle for detection using electromagnetic waves. Research performed over the past 25 years confirms that malignant breast cancer tumors show sharp variation in their dielectric properties where the value of the permittivity and conductivity of the tumor tissues are higher than normal (healthy) tissues.^{8,9} In the most comprehensive study of normal, benign and malignant breast tissue by Lazebnik et al,^{10,11} exhaustive measurements were performed to conclude that due to the complex network of glandular adipose and fibroconnective tissue in the breast, there is significant heterogeneity of dielectric properties in normal breast tissue. More specifically, the study showed that the contrast in the microwaves dielectric properties between malignant and normal adipose-dominated tissues in the breast is considerable reaching as large as 10 to 1, while the contrast in the microwaves dielectric properties between malignant and normal glandular/fibroconnective tissues in the breast is very low.

MI can be classified into two categories: tomography^{12,13} and radar-based techniques.^{13,14} Microwave tomography is based on illuminating the breast tissue to reconstruct the dielectric properties of the breast by using inverse scattering techniques that results in ill-posed nonlinear inverse problems¹⁵ which are computationally expensive and suffer from other limitations including resolution and accuracy.¹⁶⁻¹⁸ The Dartmouth group has developed the first near-field microwave imaging system that illuminates the breast with 16 monopole antennas using a circular array operating in the frequency range of 300-1000 MHz.¹⁹ Radar based modalities, on the other hand, are based on solving the forward scattering problem where the scattered signal from the breast is analysed to identify the presence of tumors.³ An example of radar-based system was developed by Klemm et al.²⁰⁻²²

### SUMMARY OF THE INVENTION

The tumor detection modality introduced in the present invention is based on two findings. The first is the aterial and topological symmetry between the two healthy breasts of a woman.^{29,30} The second is the unlikelihood that a woman would develop breast tumors in both breasts at the same time and the unlikelihood that a woman would develop identical breast tumors in both breasts at the same time.³⁰ The detection modality introduced here calls for the detection test to employ two identical probes positioned symmetrically with respect to the two breasts. The responses of the two probes are then recorded (phase and magnitude of the reflection coefficients) and processed using the PCA method to decide whether or not a tumor is present. If the response of the probes from both breasts are identical, then the woman breasts are most likely free of tumors; otherwise, there is a likelihood of the presence of a tumor that can be either benign or cancerous.

The present invention uses an electrically-small single element probe with an ultra-narrow frequency response. The shift in the magnitude and phase of the reflection coefficient of the probe caused by the presence of a tumor existing inside a human breast is used as the primary detection technique. The primary objective behind the present invention is to provide an alternative detection technique that is not only reliable for early stage cancer detection but especially inexpensive, comfortable, non-ionizing and highly accessible to a wide sector of populations in different countries. Therefore, reliability of detection and low cost are two cornerstones of the new modality proposed in the present invention. The present invention emphasizes that the proposed detection technique focuses on identifying the presence of breast tumors rather than providing an image from which the presence of a tumor can be determined.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1A shows a schematic view of the probe and the location of the matching network elements;

FIG. 1B shows a photo of the fabricated probe and the matching network;

FIG. 2 shows response of the probe over the 100-300 MHz frequency band;

FIG. 3A shows simulation setup for ultra-narrow band probe with a stand-off distance of 5 mm from a 3D realistic numerical breast phantom model;

FIG. 3B shows the embedded tumor in the numerical breast phantom;

FIG. 4A shows the probe response for normal breast tissue and breast tissue with different tumor sizes of 9 mm, 13 mm and 17 mm using the PCA feature extraction method based on shift in magnitude of reflection coefficient of the probe;

FIG. 4B shows the probe response for normal breast tissue and breast tissue with different tumor sizes of 9 mm, 13 mm and 17 mm using the PCA feature extraction method based on shift in phase of reflection coefficient of the probe;

FIG. 5A shows experiment setup of the ultra-narrow band probe with chicken breast;

FIG. 5B shows experiment setup of the ultra-narrow band probe with two slices of meat;

FIG. 6A shows a schematic diagram of the experimental procedures for horizontal scan;

FIG. 6B shows a schematic diagram of the experimental procedures for vertical scan;

FIG. 7A shows extracted and analyzed experiment results for the magnitude of S₁₁ of a chicken breast with and without an embedded metallic sphere of size 9 mm for all the horizontal scans (FIG. 6A) and a stand-off distance of 5 mm (FIG. 6B);

FIG. 7B shows extracted and analyzed experiment results for the magnitude of S₁₁ using PCA of a chicken breast with and without an embedded metallic sphere of size 9 mm for all the horizontal scans (FIG. 6A) and a stand-off distance of 5 mm (FIG. 6B);

FIG. 7C shows extracted and analyzed experiment results for the phase of S₁₁ of a chicken breast with and without an embedded metallic sphere of size 9 mm for all the horizontal scans (FIG. 6A) and a stand-off distance of 5 mm (FIG. 6B);

FIG. 7D shows extracted and analyzed experiment results for the phase of S₁₁ using PCA of a chicken breast with and without an embedded metallic sphere of size 9 mm for all the horizontal scans (FIG. 6A) and a stand-off distance of 5 mm (FIG. 6B);

FIG. 8A shows extracted and analyzed experiment results for the magnitude of S₁₁ of a chicken breast with and without an embedded metallic sphere of size 9 mm for all the horizontal scans (FIG. 6A) and a stand-off distance of 10 mm (FIG. 6B);

FIG. 8B shows extracted and analyzed experiment results for the magnitude of S₁₁ using PCA of a chicken breast with and without an embedded metallic sphere of size 9 mm for all the horizontal scans (FIG. 6A) and a stand-off distance of 10 mm (FIG. 6B);

FIG. 8C shows extracted and analyzed experiment results for the phase of S₁₁ of a chicken breast with and without an embedded metallic sphere of size 9 mm for all the horizontal scans (FIG. 6A) and a stand-off distance of 10 mm (FIG. 6B);

FIG. 8D shows extracted and analyzed experiment results for the phase of S₁₁ using PCA of a chicken breast with and without an embedded metallic sphere of size 9 mm for all the horizontal scans (FIG. 6A) and a stand-off distance of 10 mm (FIG. 6B);

FIG. 9A shows experiment results of using three different sizes of metallic spheres of size 9 mm, 13 mm and 17 mm embedded in the chicken breast using PCA with a stand-off distance of 5 mm for magnitude of S₁₁ using PCA;

FIG. 9B shows experiment results of using three different sizes of metallic spheres of size 9 mm, 13 mm and 17 mm embedded in the chicken breast using PCA with a stand-off distance of 5 mm for phase of S₁₁ using PCA;

FIG. 9C shows experiment results of using three different sizes of metallic spheres of size 9 mm, 13 mm and 17 mm embedded in the chicken breast using PCA with a stand-off distance of 10 mm for magnitude of S₁₁ using PCA;

FIG. 9D shows experiment results of using three different sizes of metallic spheres of size 9 mm, 13 mm and 17 mm embedded in the chicken breast using PCA with a stand-off distance of 10 mm for the phase of S₁₁ using PCA;

FIG. 10A shows experiment results of using three different sizes of metallic spheres of size 9 mm, 13 mm and 17 mm embedded in a slice of beef using PCA with a stand-off distance of 5 mm for magnitude of S₁₁ using PCA;

FIG. 10B shows experiment results of using three different sizes of metallic spheres of size 9 mm, 13 mm and 17 mm embedded in a slice of beef using PCA with a stand-off distance of 5 mm for phase of S₁₁ using PCA;

FIG. 10C shows experiment results of using three different sizes of metallic spheres of size 9 mm, 13 mm and 17 mm embedded in a slice of beef using PCA with a stand-off distance of 10 mm for magnitude of S₁₁ using PCA;

FIG. 10D shows experiment results of using three different sizes of metallic spheres of size 9 mm, 13 mm and 17 mm embedded in a slice of beef using PCA with a stand-off distance of 10 mm for the phase of S₁₁ using PCA;

FIG. 11A shows experiment results of using three different sizes of metallic spheres of size 9 mm, 13 mm and 17 mm embedded in a mixture of a slice of beef and chicken breast using PCA with a stand-off distance of 5 mm for magnitude of S₁₁ using PCA;

FIG. 11B shows experiment results of using three different sizes of metallic spheres of size 9 mm, 13 mm and 17 mm embedded in a mixture of a slice of beef and chicken breast using PCA with a stand-off distance of 5 mm for phase of S₁₁ using PCA;

FIG. 11C shows experiment results of using three different sizes of metallic spheres of size 9 mm, 13 mm and 17 mm embedded in a mixture of a slice of beef and chicken breast using PCA with a stand-off distance of 10 mm for magnitude of S₁₁ using PCA, and

FIG. 11D shows experiment results of using three different sizes of metallic spheres of size 9 mm, 13 mm and 17 mm embedded in a mixture of a slice of beef and chicken breast using PCA with a stand-off distance of 10 mm for the phase of S₁₁ using PCA.

### DETAILED DESCRIPTION OF PREFERRED EMBODIMENTS

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention.

The present invention presents an alternative microwave modality for breast tumor detection using a single probe for each of the two human female breasts. The sensing mechanism is simple and has the advantage of providing portability and comfort for the user. The present system has the ability of sensing a tumor buried inside breast tissues. The high sensitivity of the probe and method can detect the presence of a tumor inside a breast having inhomogeneous dielectric variations. The feature extraction algorithm, the principle component analysis (PCA), is used to enhance the changes in the scattering parameters of the probe, which aid in distinguishing between a healthy and tumorous breast.

The sensing modality conceived here is sharply different from other microwaves-based modalities that were based on reconstructing the dielectric properties profile of the breast. The modality presented here is fundamentally based on the complex interaction between the near-field of an electrically-small probe and the highly heterogeneous human female breast. The overall detection schemed is intended to provide women with regular initial stage breast cancer screening that is portable and comfortable. In this work, the feasibility of the concept was demonstrated by using human female breast phantoms and experimentally using chicken and beef tissues. Future work will focus on replacing the measurements' devices with compact circuits to ensure full portability.

The present invention comprises of a pair of probes and a method of analyzing the signal received from the probes.

The probe is a dipole antenna, small enough to confine most of the near-field radiation into the breast tissue to increase its sensitivity to variation in the dielectric properties of the tissues that are interrogated. Other types of antennas, including but not limited to loops or even patch antennas, can be used.

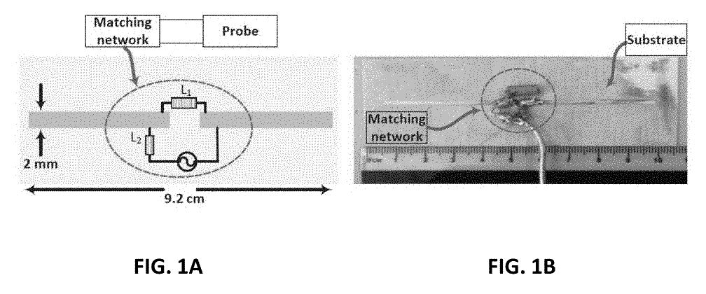

The present probe is designed as a printed dipole of length 92 mm and trace width of 2 mm hosted on an R04003 Rogers material with a thickness of 1.52 mm and a dielectric substrate of a relative permittivity of εᵣ=3.38, as shown in FIGS. 1A and 1B. The electrical length of the dipole is λ/12, (where λ is the wavelength in free space) which makes its radiation efficiency very low implying a flat, or near unity reflection coefficient (S₁₁) response, which in turn, makes it highly insensitive to breast material changes. To enable the probe to be highly sensitive, the probe must be made of an efficient radiator, which leads to a highly defined ultra-narrow band-stop filter response by using a lossless matching network.

For sufficient field penetration into the breast tissue, the frequency of operation must be such that the few skin depths cover a significant portion of the breast volume. Without loss of generality.

The frequency of operation is selected to be 200 MHz which ensures sufficient field penetration in the human female breast.

The network was designed using the full-wave numerical simulation tool CST Microwave Studio.²⁴ The optimized matching network consisted of a series and parallel inductors having inductances of 0.36 μH and 0.49 μH, respectively with specific placement of the elements as shown in FIG. 1A. The values of the inductors and their location and orientation in the present invention were optimized by CST.

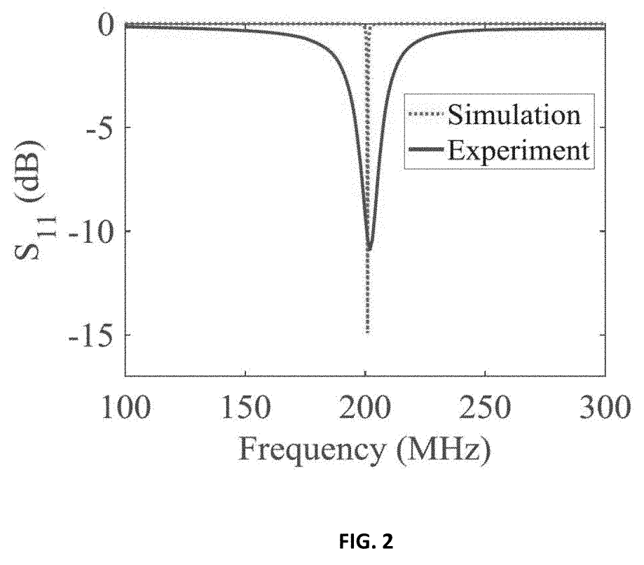

The probe is fabricated and tested yielding strong agreement between the measurements and the simulations as can be observed from FIG. 2. The broadening of the bandwidth in the measurements arises from the dispersive nature of the material and circuit elements used in the construction of the probe. Specifically, the agreement is very strong for the resonance frequency. Particularly, the dispersive nature of all materials involved in the fabrication of the probe (for instance, the inductance of real inductors is frequency dependent whereas the simulated ones have frequency independent inductance). The slight broadening of the bandwidth of the fabricated (real-world) probe in comparison with the ideal (numerical) probe has the advantage of providing additional features that enhance the detection discrimination when using the principle component analysis as shown in FIG. 2.^{14, 23}

Next, the PCA method is employed to reduce the dimensionality of the problem by implementing a vector space transform.³¹ The objective of PCA is to extract critical information from the frequency response data set and to express this information as a set of orthogonal variables called principal components.³² The eigen-decomposition of positive semi-definite matrices and the singular value decomposition of rectangular matrices are used for finding the principal components.³² Thus, via mathematical projection, high dimensional original datasets can be reduced to small number of variables without losing much of the original information to analyze trends, patterns and outliers.³¹

The PCA classification algorithm involves two steps: (1) Extraction of the reflection coefficient of each recorded scan at the different stand-off distances and tumor sizes. (2) Using MATLAB which employed the PCA algorithm to show 2D plots that indicate whether or not a tumor is present.

The two feature vectors used in the present data analysis before using the PCA method are the magnitude and phase of the reflection coefficient of the probe. The both features are a function of the operating frequency. The two features' discrete points were obtained in the simulation and measurement for a span containing 201 points and a frequency range of 100 MHz to 300 MHz. This sets the dimension of each feature to 201 rows and 2 columns. PCA is projection and dimensionality-reduction method which aims to find a small number of “directions” in input space that explain variation in input data; re-represent data by projecting along those directions. PCA search for orthogonal directions in space with the highest variance projection data onto this subspace structure of data vectors is encoded in sample covariance. The procedure to obtain 1st and 2nd PCA values from magnitude and phase of the reflection coefficient of the probe as the following:
1—Calculate the mean;
2—Centering the data (subtract the sample mean from each variable;

$$C = \frac{1}{N} {\sum }_{n = 1}^{N} ( {x}^{( n )} - \overline{x} ) {( {x}^{( n )} - \overline{x} )}^{T}$$

3—Calculate the covariance matrix, and
4—Find the M eigenvectors with largest eigenvalues of C: these are the principal components PCA1 is the highest eigen value and PCA2 is the second.

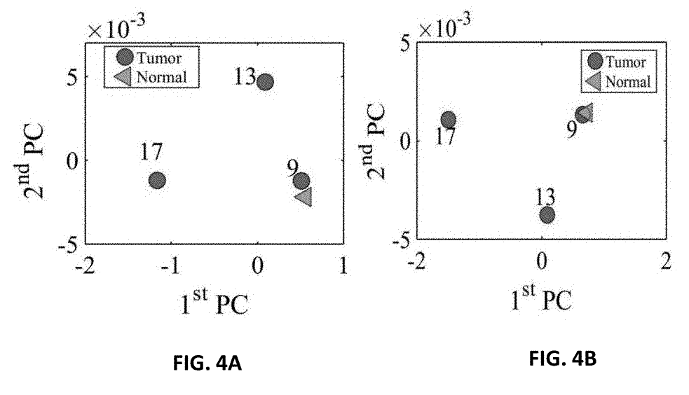

Once the frequency response of the two probes are recorded (mimicking the scenario where a single probe is used for each of the two breasts), the PCA feature extraction method is applied for both the healthy and the tumorous cases. The results of FIG. 4A for the magnitude and FIG. 4B for the phase show that the probe is capable of detecting breast tumors as small as 9 mm. Clearly, the probe is more sensitive to larger tumor sizes as indicated by the more pronounced separation between the cases with and without tumors. We also observe that for the 9 mm tumor, the magnitude response was more effective than the phase response in the sense of providing higher discrimination.

A PCA analyzer provides a spatial separation between the normal and tumorous cases. As the tumor size increases, the separation between the normal and the tumorous case increases, which indicates a more pronounced detection. Moreover, the separation between the cases with and without the tumor is more pronounced for the magnitude as compared to the phase of the S₁₁ of the probe, which is similar to the behavior observed when interrogating the numerical breast phantom.

The present probe is tested both using a simulated breast phantom and actual tests to test its ability to detect the presence of tumor cells.

In the phantom breast test, the anonymous breast MRI datasets are used, which were obtained from the University of Wisconsin online repository to build the breast phantom in the computer simulation technology (CST).²⁴⁻²⁶ The phantom is an anatomically realistic three dimensional (3D) numerical model with dielectric properties derived from the T1-weighted MRI images using a piecewise-linear mapping between MRI voxel intensity and the dielectric properties of the breast. the American College of Radiology (ACR) defines four categories of breast composition according to the radiographic density of breast fibrous and glandular tissues (table. 1).²⁷

TABLE 1
The four classes of numerical breast phantoms
according to their radiographic density.²⁷

| Class | Name of Phantom | Percentaqe of Glandular Tissue |
|---|---|---|
| 1 | Mostly fat | <25% |
| 2 | Scattered fibroglandular | 25%-50% |
| 3 | Heterogeneously dense | 51%-75% |
| 4 | Very dense | >75% |

Without loss of generality, the model used in this work is the Heterogeneously Dense Breast ID: 062204″ ACR classification: Class 3. The model has 0.5×0.5×0.5 resolution with 219×243×273 voxels. After processing the available data in Matlab, the data which includes the breast volume and the single Cole-Cole model for the frequency-dispersive tissues' properties were used to construct a numerical model in CST as:²⁸

$$ɛ ( ω ) = {ɛ}^{′} ( ω ) - j {ɛ}^{″} ( ω ) = {ɛ}_{∞} + \frac{Δɛ}{1 + {( j ωτ )}^{1 - α}} + \frac{{σ}_{s}}{j {ωɛ}_{0}}$$

where ω is the angular frequency, ε′(ω) is the frequency-dependant dielectric constant, ε″(ω) is the frequency-dependant dielectric losses and ε₀ is the free space permittivity. The ε_{∞}, σₛ, τ and α are the Cole-Cole model parameters obtained from the clinical experimental data. The breast model has the physical shape of a real human female breast. Moreover, with a high resolution of 0.5 mm, the model also includes the following eight tissue types: skin, muscle, glandular-1,2,3, and fat-1,2,3.²⁷

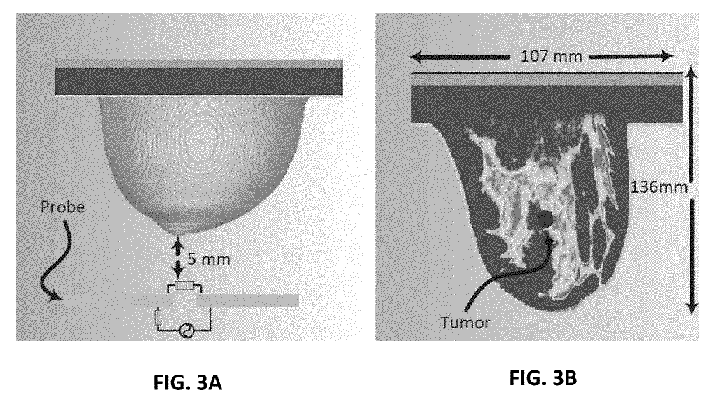

The probe is first calibrated with the numerical realistic breast phantom where the proposed probe is placed at a distance of 5 mm away from the healthy breast phantom as shown in FIG. 3A. The magnitude and phase of the reflection coefficient of the probe were then recorded at 109 different frequencies spanning the range 160 to 220 MHz. Next, a tumor is inserted inside the same breast phantom with three different diameter sizes namely: 9 mm, 13 mm, and 17 mm, as shown in FIG. 3B. The tumor's dielectric properties were obtained from cancer surgery as documented in¹¹. The magnitude and phase information were then recorded for the breast with the tumor. The data is then analysed with and without the tumor to decide whether or not a tumor is present.

The probe standalone in the present invention has a certain resonance frequency. However, when the probe is brought close to the breast the resonance is shifted due to the effect of the breast on the near field of the probe or the input impedance of the probe. This shift will exist whether there is a tumor or not. That's why the present invention compares the scattering parameters with a healthy breast Vs. breast with tumor and not with the probe alone. This is to make sure that the resonance frequency, the present invention is analysing is after adding the effect of the breast.

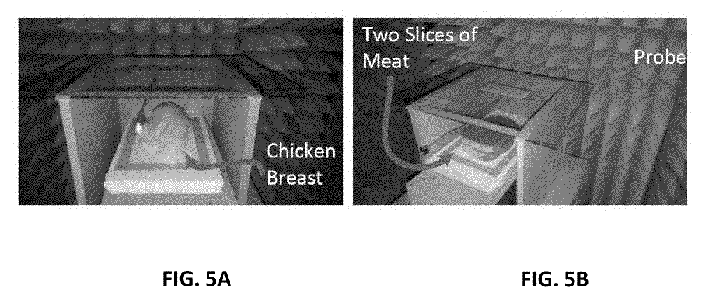

In addition, an experiment was carried out in an environment that have resemblance to female breast tissues. The experiment setup consisted of the electrically-small dipole probe, a keysight 8.5 GHz VNA, metallic spheres with different diameter sizes (mimicking a tumor), chicken breast tissue and a slice of a beef steak. The experiment apparatus was contained in a wooden box with an opening in one of the box sides to allow for easily placing and changing the position of the material under test inside the box as shown in FIGS. 5A and 5B. The experiment was performed inside an anechoic chamber to ensure that no energy is bouncing back from the surrounding environment and effecting the measured probe response.

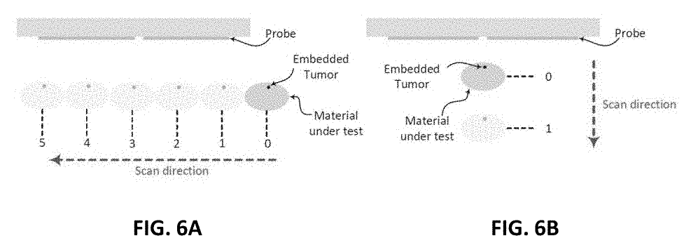

Initially, the material under test is scanned horizontally at 6 different positions as shown in FIG. 6A. The main reason for performing such a scan is to determine the location where the probe sensitivity is highest (highest sensitivity refers to the highest shift experienced by the magnitude and phase of the S₁₁ of the probe when compared to the original case). For instance, the S₁₁ of the probe in the presence of an object placed at the edge of the dipole probe compared to that of the same object placed at the center of the probe is quite different. This is mainly due to the fact that a high electric field is confined within the gap of the dipole at the resonance frequency. When an object is placed in the vicinity of the gap, the probe's response experiences higher shift in both the magnitude and phase than the case where the object is placed further away from the gap. Hence, the probe is highly sensitive to an object placed above or close to the gap. Once the position of highest sensitivity was found, FIG. 6A, the probe response was then recorded for this high sensitivity position. Although, the middle of the probe experienced the highest sensitivity, other locations away from the gap have a reasonable shift which can lead to a successful differentiation between the normal and the tumor breast tissue. Therefore, in a real-world detection scenario, the patient can position the probe in any location in front of the breast to perform the self examination.

Next, three different tests were performed: 1) with chicken breast, 2) a slice of beef, and 3) a hybrid of both. In each test, two main variables were changed. First, multiple metallic spheres, which will be embedded inside the material under test with different diameter sizes were used. Three different diameter sizes were used: 9 mm, 13 mm, and 17 mm to test the ability of the probe to detect various tumor sizes. For each tumor size, two different stand-off vertical distances were used as shown in FIG. 6B. In the experiment, the scattering parameters seen from the port of the probe were recorded for both the phase and the magnitude. The phase and magnitude of the scattering parameters were analyzed with and without the PCA method for two vertical distances of 5 mm and 10 mm as shown in FIG. 6B.

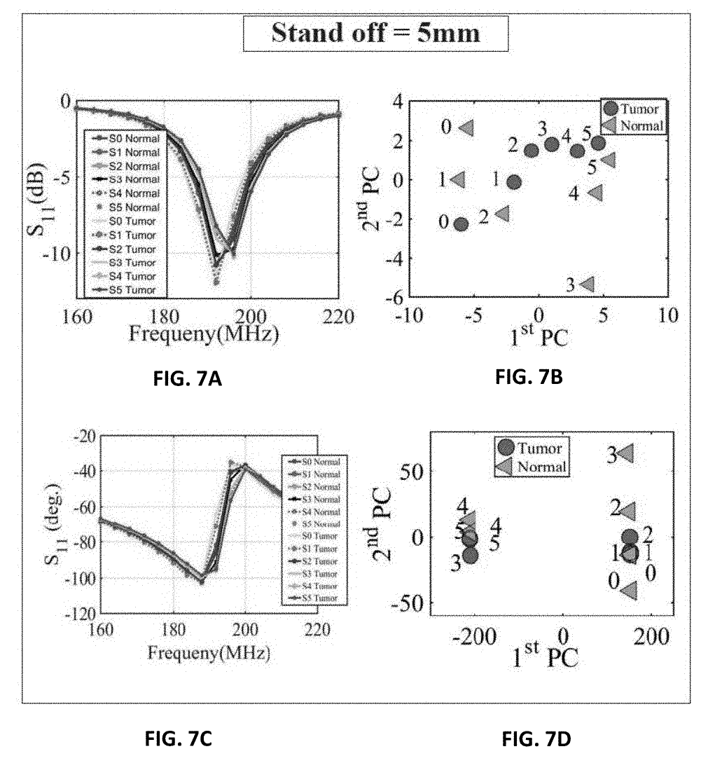

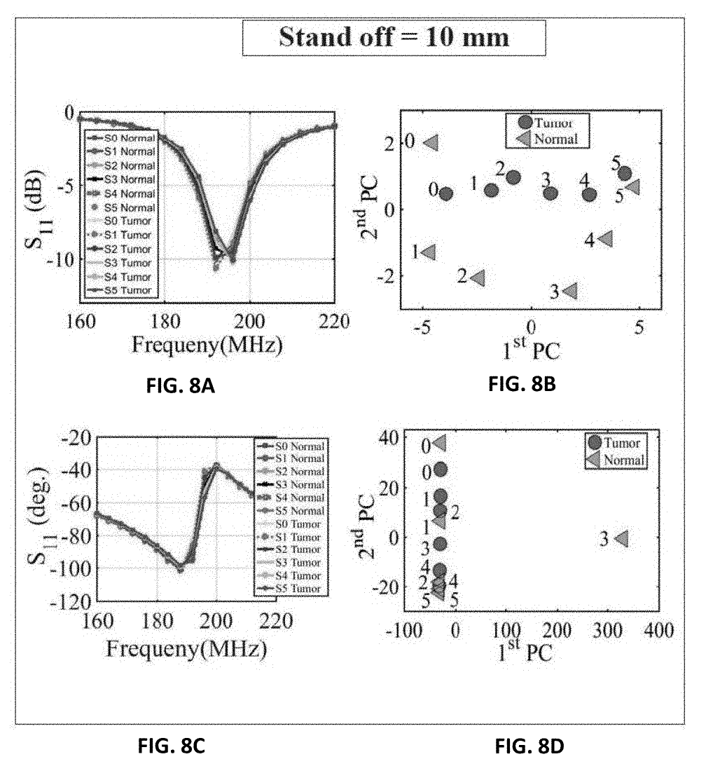

The results are presented in FIGS. 7A, 7B, 7C, 7D and FIGS. 8A, 8B, 8C and 8C. It is evident from the results that the plots of the reflection coefficient of the probe in the presence of the metallic sphere for both the magnitude and phase as shown in FIG. 7A, FIG. 7C, FIG. 8A and FIG. 8C, experience a very slight shift that is hard to detect for the various locations labelled 0-5 (see FIG. 6A). Here, the difficulty in detecting the difference between the tumours breast compared to a healthy one refers to the fact that a human eye cannot easily distinguish the change in either the magnitude or the phase of the probe. Additionally, by direct inspection of the magnitude and phase of the reflection coefficient, it is extremely difficult, if not impossible, to differentiate the healthy breast from the tumorous one, even if the process is automated, without additional classification procedure. However, when the data were plotted using the PCA method as shown in FIG. 7B, FIG. 7D, FIG. 8B and FIG. 8D, a clear and obvious distinction is observed between the healthy breast tissue (no sphere) compared to that of the malignant case (with an embedded sphere).

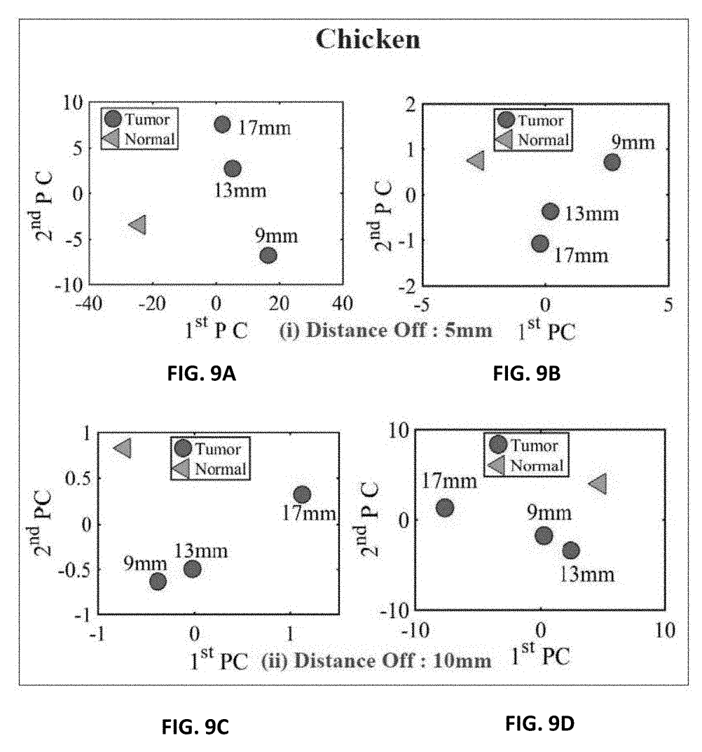

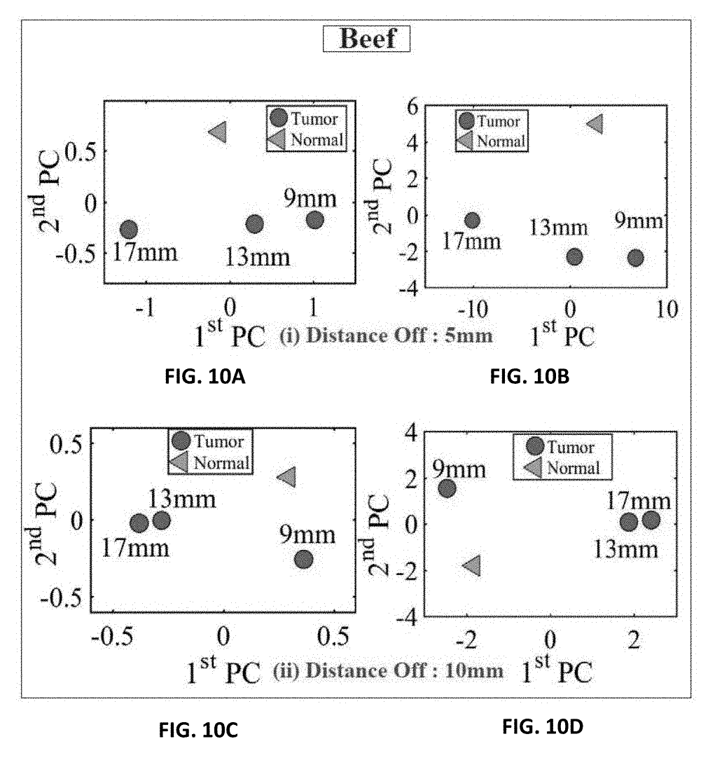

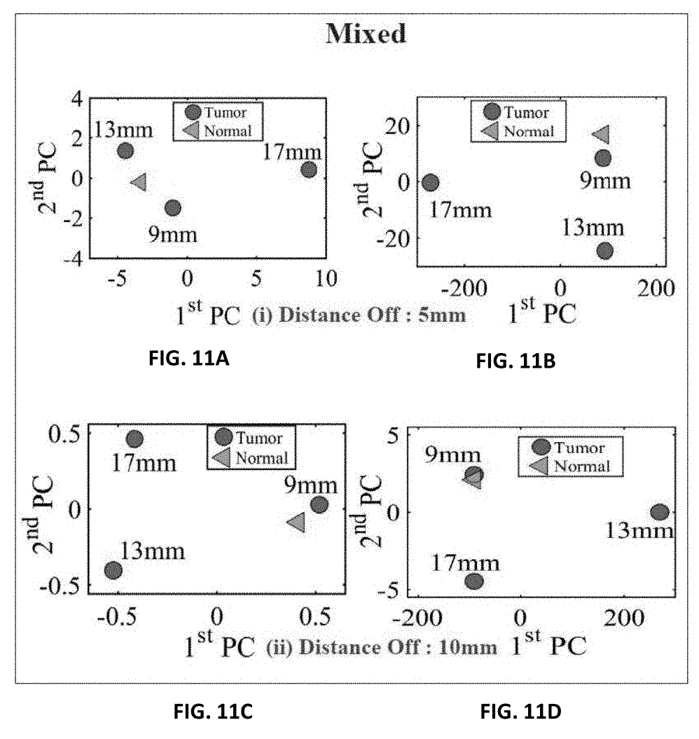

For the first experiment, a chicken breast which includes skin, fat and meat was used as a testing material. The chicken was positioned at the fixed horizontal distance labeled 3 in FIG. 6A. Then the magnitude and phase of the probe were extracted with two stand-off distances, namely 5 mm and 10 mm, as shown in FIG. 6B. The experiment was then repeated after inserting a metallic sphere inside the same chicken breast. Please note that during the experiment, we labeled the location where the spheres were embedded inside the chicken to ensure a fare and identical comparison. The results obtained from this experiment is shown in FIGS. 9A, 9B, 9C and 9D. The second experiment was conducted with a slice of meat instead of chicken to test the strength of the probe to detect the variation inside the material with different electrical properties. Similarly, the test was done for different stand-off distances and different sphere sizes. The results obtained from this case is shown in FIGS. 10A, 10B, 10C and 10D. In the last experiment, a hybrid medium consisting of a chicken breast sandwiched between two slices of meat were used to test material with higher degree of heterogeneity than the previous two cases. The results are plotted in FIGS. 11A, 11B, 11C and 11D.

The present system is also tested by numerical simulations using the full-wave electromagnetic simulator CST Microwave Studio.²⁴ In the simulation setup, a realistic breast phantom and electrically-small probe was created using MATLAB³³ and CST. The University of Wisconsin-Madison online repository provided a database of anatomically realistic numerical breast phantoms that can be used in CST. Each phantom has three ASCII text files including breastlnfo.txt, mtype.txt, and pval.txt.³⁴ The breastlnfo.txt file provides the basic information about the numerical phantom, such as breast ID: 062204, dimensions of the grid in units of grid cells [“s1”, “s2”, “s3”], and the classification of breast composition' Heterogeneously Dense'. The mtype.txt file contains data of different voxel in the grid. The pval.txt file gives dielectric properties data for each voxel of the breast phantom.²⁶ In order to create a realistic breast phantom model in CST, three files were built as follows: (1) Vox file (ascii), (2) Material .txt file, and (3) Material property .txt or binary file. In the first step, we generated the material file and material property which contains all the data of the dielectric properties of all tissues of the breast using a single-pole Cole-Cole model in MATLAB. Then we created the vox file which contains the material properties. The final step involved importing the vox file into CST.

The present invention can operate using different waves other than a microwave. The device can be scaled to operate at a frequency that is either higher of lower than the one selected for this study. However, here the present invention is focusing on microwave frequencies. Specifically, those frequencies at the lower end of the microwave spectrum. (e.g 100 MHz to 2000 MHz).

The device's probe operate based on the principle of resonance. Resonance frequency depends greatly on the dimensions of the probe and more specifically on the length of the probe. If high frequency is desired, the length of the probe can be decreased. Conversely, if lower frequencies are desired, longer lengths of the probe can be used. The main difference is the ability of an electromagnetic wave to penetrate the targeted object to be detected. The lower the frequency the better the penetration will be. However, this comes to the price of larger size. Additionally, one can operate the probe at a frequency that is different from the resonance frequency by matching the probe at the other desired frequency. The probe in this device is an example of such scenario.

In the present invention, the probe can be located in a close proximity to the breast. The preferred distance is between 1 mm to 50 mm from the tip of the breast to the probe surface. However, the probe can be designed to operate at distances of less than 1 mm as well.

### REFERENCES

1. Society, A. C. Cancer facts and figures. 2017 @ONLINE (2017). URL https://www.cancer.org/cancer/breast-cancer/about/how-cornmon-is-breast-cancer.html/.
2. Berg, W. A. et al. Diagnostic accuracy of mammography, clinical examination, us, and mr imaging in preoperative assessment of breast cancer. Radiology 233, 830-849 (2004).
3. Fear, E. C., Li, X., Hagness, S. C. & Stuchly, M. A. Confocal microwave imaging for breast cancer detection: Localization of tumors in three dimensions. IEEE Transactions on Biomedical Engineering 49, 812-822 (2002).
4. on Breast Cancer Screening, I. U. P. et al. The benefits and harms of breast cancer screening: an independent review. The Lancet 380, 1778-1786 (2012).
5. Barh, D. Omics Approaches in Breast Cancer: Towards Next-Generation Diagnosis, Prognosis and Therapy (Springer,2014).
6. Moore, A. The practice of breast ultrasound: Techniques, findings, differential diagnosis (2010).
7. Orel, S. G. & Schnall, M. D. Mr imaging of the breast for the detection, diagnosis, and staging of breast cancer. Radiology 220, 13-30 (2001).
8. Foster, K. R. & Schwan, H. P. Dielectric properties of tissues. Handbook of biological effects of electromagnetic fields 2, 25-102 (1995).
9. Gabriel, S., Lau, R. & Gabriel, C. The dielectric properties of biological tissues: Ii. measurements in the frequency range 10 hz to 20 ghz. Physics in medicine and biology 41, 2251 (1996).
10. Lazebnik, M. et al. A large-scale study of the ultrawideband microwave dielectric properties of normal breast tissue obtained from reduction surgeries. Physics in medicine and biology 52, 2637 (2007).
11. Lazebnik, M. et al. A large-scale study of the ultrawideband microwave dielectric properties of normal, benign and malignant breast tissues obtained from cancer surgeries. Physics in medicine and biology 52, 6093 (2007).
12. Semenov, S. Microwave tomography: review of the progress towards clinical applications. Philosophical Transactions of the Royal Society of London A: Mathematical, Physical and Engineering Sciences 367, 3021-3042 (2009).
13. Bond, E. J., Li, X., Hagness, S. C. & Van Veen, B. D. Microwave imaging via space-time beamforming for early detection of breast cancer. IEEE Transactions on Antennas and Propagation 51,1690-1705 (2003).
14. Zastrow, E., Davis, S. K. & Hagness, S. C. Safety assessment of breast cancer detection via ultrawideband microwave radar operating in pulsed-radiation mode. Microwave and Optical Technology Letters 49, 221-225 (2007).
15. Conceic, a\tilde{ }o, R. C., Mohr, J. J. & O'Halloran, M. An introduction to microwave imaging for breast cancer detection (Springer, 2016).
16. Bindu, G. N. et al. Active microwave imaging for breast cancer detection. Progress In Electromagnetics Research 58, 149-169 (2006).
17. Bindu, G. et al. Detection of dielectric contrast of breast tissues using confocal microwave technique. Microwave and Optical Technology Letters 48, 1187-1190 (2006).
18. Delbary, F., Brignone, M., Bozza, G., Aramini, R. & Piana, M. A visualization method for breast cancer detection using microwaves. SIAM Journal on Applied Mathematics 70, 2509-2533 (2010).
19. Meaney, P. M., Fanning, M. W., Li, D., Poplack, S. P. & Paulsen, K. D. A clinical prototype for active microwave imaging of the breast. IEEE Transactions on Microwave Theory and Techniques 48, 1841-1853 (2000).
20. Klemm, M., Craddock, I. J., Leendertz, J. A., Preece, A. & Benjamin, R. Radar-based breast cancer detection using a hemispherical antenna array—experimental results. IEEE transactions on antennas and propagation 57, 1692-1704 (2009).
21. Li, X. & Hagness, S. C. A confocal microwave imaging algorithm for breast cancer detection. IEEE Microwave and wireless components letters 11, 130-132 (2001).
22. Craddock, I., Klemm, M., Leendertz, J., Preece, A. & Benjamin, R. Development and application of a uwb radar system for breast imaging. In Antennas and Propagation Conference, 2008. LAPC 2008. Loughborough, 24-27 (IEEE, 2008).
23. Winters, D. W., Bond, E. J., Van Veen, B. D. & Hagness, S. C. Estimation of the frequency-dependent average dielectric properties of breast tissue using a time-domain inverse scattering technique. IEEE Transactions on Antennas and Propagation 54, 3517-3528 (2006).
24. CST. Computer simulation technology. cst computer simulation technology ag@ONLINE (2017). URL http://www.CST.com.
25. UWCEM. Breast phantom repository@ONLINE (2017). URL http://uwcem.ece.wisc.edu/phantomRepository.html.
26. Zastrow, E. et al. Database of 3d grid-based numerical breast phantoms for use in computational electromagnetics simulations. Department of Electrical and Computer Engineering University of Wisconsin-Madison (2008).
27. Zastrow, E. et al. Development of anatomically realistic numerical breast phantoms with accurate dielectric properties for modeling microwave interactions with the human breast. IEEE Transactions on Biomedical Engineering 55, 2792-2800 (2008).
28. Lazebnik, M. et al. A large-scale study of the ultrawideband microwave dielectric properties of normal breast tissue obtained from reduction surgeries. Physics in medicine and biology 52, 2637 (2007).
29. Gazhonova, V. 3D Automated Breast Volume Sonography: A Practical Guide (Springer, 2016).
30. Chen, J.-H. et al. Response of bilateral breasts to the endogenous hormonal fluctuation in a menstrual cycle evaluated using 3d mri. Magnetic resonance imaging 31, 538-544 (2013).
31. Richardson, M. Principal component analysis. URL: http://people.maths.ox.ac.uk/richardsonm/SignalProcPCA.pdf (last access: 3.5. 2013). Ale's Hladnik Dr., Ass. Prof, Chair of Information and Graphic Arts Technology, Faculty of Natural Sciences and Engineering, University of Ljubljana, Slovenia ales. hladnik@ ntf.uni-lj.si (2009).
32. Abdi, H. & Williams, L. J. Principal component analysis. Wiley interdisciplinary reviews: computational statistics 2, 433-459 (2010).
33. MATLAB. Matlab program@ONLINE (2017). URL https://www.mathworks.com/store?s_eid=ppc_29850095842&q=matlab.
34. of Wisconsin System, U. University of wisconsin cross-disciplinary electromagnetics laboratory@ONLINE (2017). URL http://uwcem.ece.wisc.edu/phantomRepository.html.

What is claimed is:

1. A method to detect breast tumor, said method comprising:
a) placing a transceiver/probe operating in a microwave regime at a predefined distance with respect to a first breast;
b) applying a microwave signal to the first breast;
c) receiving and recording a first scattered signal from the first breast;
d) placing a transceiver/probe operating in a microwave regime at said predefined distance with respect to a second breast;
e) applying a microwave signal to the second breast;
f) receiving and recording a second reflected signal from the second breast;
g) applying a principle component analysis (PCA) on the first and the second scattered signals to express a set of orthogonal variables and to identify a first PCA point and a second PCA point on a PCA diagram corresponding to the first and the second breasts, respectively;
h) comparing the first PCA point and the second PCA point, and
i) identifying a tumorous breast if a distance exists between the first and the second PCA points on the PCA diagram.

2. The method of claim 1, wherein the scattered signal comprises of shifts in a resonance frequency and shifts in a magnitude and/or phase of a reflection coefficient.

3. The method of claim 1, wherein the transceiver/probe is operated at a resonance frequency of the probe as located in proximity of the breasts.

4. The method of claim 1, wherein an artificial intelligence method comprising of neural networks, principal component analysis and fuzzy logic is used to identify the proximity of the first PCA point and the second PCA point on the PCA diagram for the existence of a tumor.

5. The method of claim 1, wherein the predefined distance is preferably in a range between 1 mm to 50 mm from the tip of a breast to a probe surface.

6. A method to detect breast tumor, said method comprising:
a) placing a first and a second transceiver/probe operating in a microwave regime at a predefined distance with respect to a first breast and a second breast respectively;
b) applying a microwave signal to the first breast and the second breast;
c) receiving and recording a first and a second scattered signals from the first breast and the second breast respectively;
d) applying a principle component analysis (PCA) on the first and the second scattered signals to express a set of orthogonal variables and to identify a first PCA point and a second PCA point on a PCA diagram corresponding to the first and the second breasts on, respectively;
e) comparing the first PCA point and the second PCA point, and
f) identifying a tumorous breast if a distance exists between the first and the second PCA points on the PCA diagram,
whereby the proximity of the first PCA point and the second PCA point on the PCA diagram distinguishes between a healthy and a tumorous breast and whereby the method is based on material and topological symmetry between two healthy breasts and unlikelihood of developing breast tumors and/or identical breast tumors in both breasts at the same time.

7. The method of claim 6, wherein the predefined distance is preferably in a range between 1 mm to 50 mm from the tip of a breast to a probe surface.

8. The method of claim 6, wherein the scattered signal comprises of shifts in a resonance frequency and shifts in a magnitude and/or phase of a reflection coefficient.

9. The method of claim 6, wherein the transceiver/probe is operated at a resonance frequency of the probe as located in proximity of the breasts.

10. The method of claim 6, wherein an artificial intelligence method comprising of neural networks, principal component analysis and fuzzy logic is used to identify the proximity of the first PCA point and the second PCA point on the PCA diagram for the existence of a tumor.

11. A device to detect breast tumor, said device comprising:
a) a transceiver/probe operating in a microwave regime, wherein said transceiver/probe is placed once at a predefined distance with respect to a first breast and then at a predefined distance with respect to a second breast to apply a microwave signal to the first breast;
b) a receiving-recording unit to receive and record a first scattered signal and a second scattered signal from the first breast and the second breast, respectively;
c) a processor to apply a principle component analysis (PCA) on the first and the second scattered signals to express a set of orthogonal variables and to identify a first PCA point and a second PCA point on a PCA diagram corresponding to the first and the second breasts on, respectively, and comparing the first PCA point and the second PCA point for the existence of the tumor,
whereby the proximity of the first PCA point and the second PCA point on the PCA diagram distinguishes between a healthy and a tumorous breast and whereby the method is based on material and topological symmetry between two healthy breasts and unlikelihood of developing breast tumors and/or identical breast tumors in both breasts at the same time.

12. The device of claim 11, further having a display unit to show the PCA diagram.

13. The device of claim 11, wherein the transceiver/probe is made of a single element or an array of elements with single or multiple feeds.

14. The device of claim 11, wherein the transceiver/probe is selected from the groups consisting of a dipole antenna, a loop antenna or a patch antenna.

15. The device of claim 11, wherein the transceiver/probe is a printed dipole of length 92 mm and trace width of 2 mm hosted on a RO4003 Rogers material with a thickness of 1.52 mm.

16. The device of claim 11, wherein the resonance frequency of he transceiver/probe is 100-2000 MHz to ensure enough field penetration in a female breast.

17. The device of claim 11, wherein the transceiver/probe is made of an efficient radiator which lead to a highly defined ultra-narrow band-stop filter response by using a lossless matching network.
